(19) United States
(12) Patent Application Publication
(10) Pub. No.: US 2013/0131111 A1
(43) Pub. Date: May 23, 2013

(54) NMN MODULATORS FOR THE TREATMENT OF NEURODEGENERATIVE DISORDERS

(71) Applicants: Michael Coleman, Cambridge (GB); Laura Conforti, Cambridge (GB)

(72) Inventors: Michael Coleman, Cambridge (GB); Laura Conforti, Cambridge (GB)

(73) Assignee: Babraham Institute, Cambridge, Cambridgeshire (GB)

(21) Appl. No.: 13/643,997

(22) PCT Filed: Apr. 19, 2011
(86) PCT No.: PCT/GB2011/050770

§ 371 Date: Jan. 24, 2013

(30) Foreign Application Priority Data
Apr. 27, 2010 (GB) 1006961.5

(51) Int. Cl. A61K 31/4545; A61K 31/4409

(52) U.S. Cl. CPC A61K 31/4545; A61K 31/4409
USPC 514/318; 546/194; 546/306; 514/353; 536/26.8; 436/501; 435/7.92

(57) ABSTRACT

The invention relates to a nicotinamide mononucleotide (NMN) modulator useful as a neuroprotective medicament in the treatment of neurodegenerative disorders, in particular but not exclusively disorders involving axon degeneration of neuronal tissue such as Wallerian degeneration, to the use of NMN as a biomarker for axon degeneration, to a method of demonstrating axon degeneration using an NMN-based biomarker, to a diagnostic kit for detecting axon degeneration, to a method of screening for an NMN modulator, and to an NMN modulator identified using the aforementioned screening method.

### FIELD OF THE INVENTION

The invention relates to a nicotinamide mononucleotide (NMN) modulator useful as a neuroprotective medicament in the treatment of neurodegenerative disorders, in particular but not exclusively disorders involving axon degeneration of neuronal tissue such as Wallerian degeneration, to the use of NMN as a biomarker for axon degeneration, to a method of demonstrating axon degeneration using an NMN-based biomarker, to a diagnostic kit for detecting axon degeneration, to a method of screening for an NMN modulator, and to an NMN modulator identified using the aforementioned screening method.

### BACKGROUND OF THE INVENTION

Neurodegenerative diseases are characterised by a loss of viable nerve cells from either the peripheral or the central nervous system. In many cases this loss has been shown to be preceded by degeneration of the neuronal axon, which is invariably more pronounced at the distal rather than the proximal end of axonal processes. There are two models which attempt to explain this greater degree of distal axonal degeneration. The first is ‘dying back’ in which degeneration spreads retrogradely from the nerve terminals. The second is Wallerian degeneration where degeneration spreads from the site of a lesion in either direction according to the lesion type; this ultimately results in loss of the axon distal to the lesion site, leaving the proximal portion intact. Although strictly speaking Wallerian degeneration only occurs in response to physical injury of the axon, similar mechanisms operate in diseases where no such injury has occurred. The latter is referred to as ‘Wallerian-like’ degeneration. Both types of degeneration will hereinafter be jointly referred to as ‘Wallerian degeneration’.

The recently discovered WldS mouse has led to progress in the understanding of these two processes. In these animals, Wallerian degeneration occurs at a rate roughly ten times slower than in wild-type animals. Studies have shown that this mutation also delays pathologies believed to involve ‘dying back’ of axonal terminals. The WldS gene therefore provides a mechanistic link between the two models of axonal degeneration.

Axon degeneration is an area of unmet therapeutic need. It is a major cause of symptoms in motor neuron disease, glaucoma, Alzheimer's disease and multiple sclerosis. In diabetes it causes neuropathic pain and distal sensory loss, which is a leading cause of limb amputation. It is a dose-limiting side effect in cancer chemotherapy. Progressive axon degeneration due to stretch injury is the major pathology in traumatic brain injury and failure to protect white matter limits the treatment for stroke. Around half the population will suffer one or more of these disorders, which significantly reduces quality of life.

Despite the identification and characterisation of the WldS gene, progress towards understanding of the molecular trigger for Wallerian degeneration has been limited. Knowledge of this trigger could have a profound impact on the understanding of the early stages of ‘dying-back’ neurodegenerative diseases.

There is no effective treatment for axon degeneration, no means of prevention and little natural repair in the CNS, so there is a great need for new molecular targets to reduce axon degeneration.

### BRIEF DESCRIPTION OF THE FIGURES

FIG. 1 is a schematic representation of mammalian NAD⁺ metabolic pathways (modified from Hassa et al (2006) Microbiology and Molecular Biology Reviews 70(3), 789-829);

FIG. 2 describes the results of the analysis of the effect of the Nampt inhibitor FK866 upon NAD levels;

FIG. 3 describes the results of the analysis of the effect of the Nampt inhibitor FK866 upon cut neurites;

FIG. 4 describes how NMN reverts the protective effect of the Nampt inhibitor FK866 on cut neurites; and

FIG. 5 describes the results of the analysis of the effect of the Nampt inhibitor FK866 upon neurites treated with the neurotoxic chemotherapy drug vincristine and how NMN⁺ reverts the protective effect of FK866 on vincristine-treated neurites.

### DETAILED DESCRIPTION OF THE INVENTION

According to a first aspect of the invention there is provided the use of a nicotinamide mononucleotide (NMN) modulator as a neuroprotective medicament in the treatment of a neurodegenerative disorder.

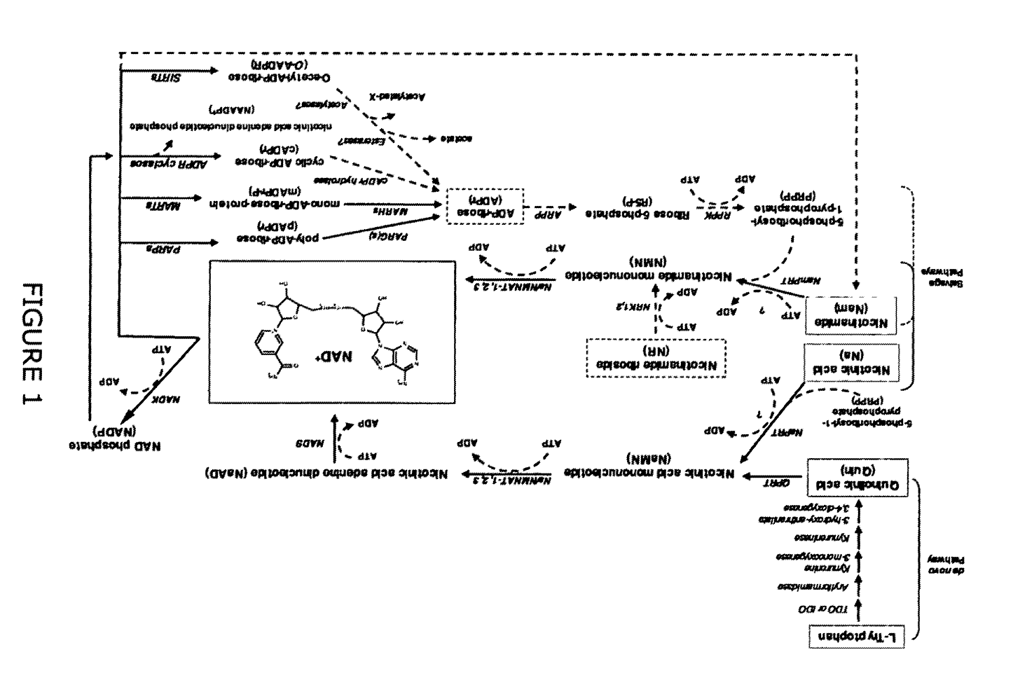

NMN (also referred to as NMN⁺) is a component in the NAD⁺ (nicotinamide adenine dinucleotide) biosynthetic salvage pathway (FIG. 1) by virtue of being a precursor for the formation of NAD⁺.

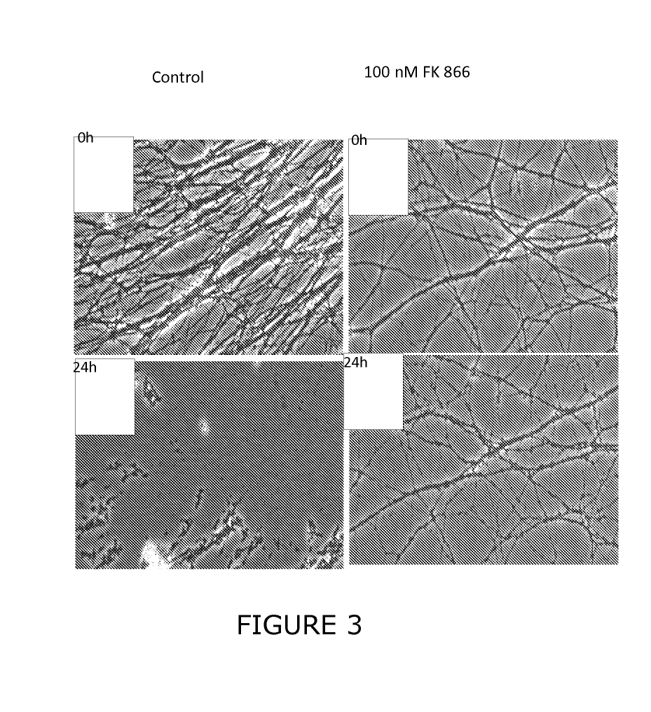

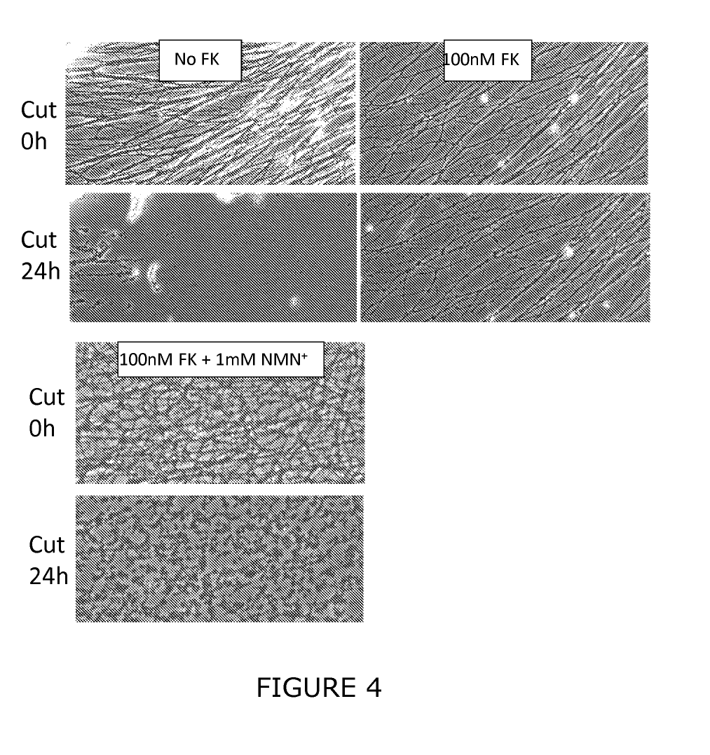

The term ‘modulator’ as used herein refers to a molecule capable of altering the intracellular levels of NMN, either directly or indirectly. In one embodiment, the modulator directly alters the intracellular levels of NMN. Data is presented herein which demonstrates that a reduction in NMN levels associated with protection of injured axons in culture (see Example 2 and FIG. 3 which show lowering of NAD⁺ levels) thus mimicking the slow Wallerian degeneration (Wld^{s}) phenotype. Furthermore, this effect was reversed when NMN was added to the culture (see Example 3 and FIG. 4). Therefore, in the present context, the goal is to decrease NMN levels. Thus, in one embodiment, the NMN modulator is an inhibitor of NMN generation, i.e. an agent capable of decreasing NMN levels.

It will be appreciated that decreasing NMN levels can be achieved through several means. For example, in one embodiment the NMN inhibitor is a Nampt inhibitor.

Nampt (EC Number 2.4.2.12; also known as nicotinamide phosphoribosyltransferase, PBEF, NAPRT or visfatin) is an essential enzyme in the NAD⁺ (nicotinamide adenine dinucleotide) biosynthetic salvage pathway, catalyzing the condensation of nicotinamide with 5-phosphoribosyl-1-pyrophosphate to yield NMN, an intermediate step in the biosynthesis of nicotinamide adenine dinucleotide (NAD⁺). From a review of the NAD pathway in FIG. 1 it is apparent that Nampt inhibition will have the effect of depleting NMN levels.

The NAD⁺ biosynthetic pathway (FIG. 1) has been well characterised in the field of cancer therapy and therefore Nampt inhibitors are well known, commercially available and have been extensively investigated. Thus, in one embodiment, the Nampt inhibitor is N-[4-(1-benzoyl-4-piperidinyl)butyl]-3-(3-pyridinyl)-2E-propenamide (FK866; K 22.175; CAS Number: 658084-64-1):

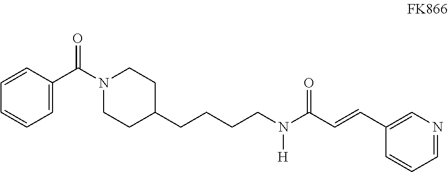 [SMILES: O=C(/C=C/c1cccnc1)NCCCCC1CCN(C(=O)c2ccccc2)CC1]

FK866 is a highly specific non-competitive inhibitor of Nampt (Kᵢ=0.4 nM), causing gradual NAD⁺ depletion (Hasmann, M., Schemainda, I. (2003) Cancer Res 63; 7436-7442).

In an alternative embodiment, the Nampt inhibitor is N-(6-chlorophenoxyhexyl)-N′-cyano-N″-4-pyridylguanidine (CHS828):

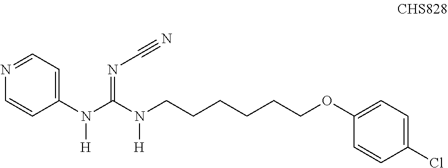 [SMILES: N#C/N=C(\NCCCCCCOc1ccc(Cl)cc1)Nc1ccncc1]

As with FK866, CHS828 is also a Nampt inhibitor and has been found to kill cancer cells by depleting NAD⁺ (Olesen, U H et al (2008) Biochemical and biophysical research communications 367(4), 799-804).

Clinical cancer studies relating to other anti-cancer chemotherapeutics have identified axon degeneration as a frequent, dose-limiting side effect (for example, Taxol, Velcade and vincristine are known to cause peripheral neuropathy), therefore, the fact the present invention has identified a neuroprotective effect with anti-cancer agents, such as Nampt inhibitors (i.e. FK866) constitutes a surprising finding.

When the use of the invention comprises a Nampt inhibitor, the Nampt inhibitor may additionally comprise nicotinic acid adenine dinucleotide (NaAD). The inventors have found that although Nampt inhibitors such as FK866 have a neuroprotective effect upon axons, such an effect does not extend to cell bodies (data not shown). It is believed that this may be a consequence of the depletion of NMN and therefore NAD. Adding NAD or NMN will avoid the harmful effect upon cell bodies but is likely to revert the axon protective effect provided by the Nampt inhibitor. By contrast, addition of an NAD raising agent, such as NaAD is likely to provide the benefit of restoring cell body viability without reverting the beneficial effect of the Nampt inhibitor.

In an alternative embodiment, the NMN modulator is a Nmnat activator.

Nmnat (Nicotinamide/nicotinate mononucleotide adenylyltransferase) is the central enzyme of the NAD⁺ (nicotinamide adenine dinucleotide) biosynthetic pathway, catalysing the formation of NAD⁺ from NMN⁺ (nicotinamide mononucleotide) and NaAD (nicotinic acid adenine dinucleotide) from NaMN (nicotinic acid mononucleotide). From a review of the NAD pathway in FIG. 1 it is apparent that Nmnat activation will have the effect of depleting NMN levels.

Three isoforms of the enzyme have been identified, expressed by three different genes in mammals: Nmnat1, Nmnat2 and Nmnat3. Other synonyms for Nmnat isoforms are KIAA0479 for the Nmnat2 protein, D4Cole1e for the gene encoding the Nmnat1 protein or Ensadin 0625 for the gene encoding the Nmnat2 protein. Nmnat2 may exist in more than one splice form, all of which are referred to here as Nmnat2. Nmnat2 appears to be mainly expressed in brain, heart and muscle tissue whereas Nmnat1 and Nmnat3 show a wider distribution pattern throughout a range of tissues. At the cellular level, Nmnat1 is most abundant in the nucleus, Nmnat2 is abundant in the Golgi complex and in vesicles within axons and Nmnat3 is abundant in mitochondria. Other subcellular locations are also possible in each case.

Data has been previously presented which shows that a knock-down of Nmnat2, a different isoform from the Nmnat1 incorporated into the protective WldS protein, induced rapid Wallerian-like degeneration. By contrast, experimental reduction in expression of Nmnat1 or Nmnat3 had no effect on the rate of axonal degeneration.

The term “Nmnat activator” as used herein refers to an agent that increases the total level of Nmnat activity in the axon. Examples of such activators include agents which: increase Nmnat protein expression; increase Nmnat delivery to axons; slow Nmnat turnover; increase concentration of potentially important enzyme co-factors; allosterically activate the enzyme; enhance substrate binding to the enzyme; enhance subcellular targeting to a key location; or increase half-life of the enzyme whether through direct interaction with Nmnat or interaction with a protein involved in its degradation.

Without being bound by theory, it is believed that the pro-survival actions of Nmnat activation and Nampt inhibition by FK866 (which synthesise and deplete NAD⁺ respectively), may exert their effect by virtue of the Nampt product NMN⁺, or a derivative, being harmful to axons. Nmnat is currently the only cytoplasmic enzyme known to use NMN⁺, so NMN⁺ could accumulate in injured or sick axons after Nmnat2 is degraded. In this instance, Wld^{S}, when present, would scavenge this NMN⁺ and FK866 would prevent Nampt producing it.

Axon protection by FK866 has been previously analysed (Sasaki et al., 2009, The Journal of Neuroscience, 29(17), 5525-5535), however, this prior study indicated that Nmnat-mediated axonal protection is not correlated with intracellular NAD⁺ levels. More critically, this prior study identified that genetic inhibition of Nampt greatly reduced neuronal NMN and NAD⁺ levels, yet did not lead to axonal protection (or degeneration) and that Nmnat mediated neuronal protection does not operate through altering neuronal NAD⁺ or NMN levels. Contrary to these findings, data is clearly presented herein which demonstrates that adding NMN⁺ to bypass Nampt consistently reverts the protective effect of the Nampt inhibitor FK866 (FIG. 4).

In a further alternative embodiment, the NMN modulator is an NMN sequestering agent. Without being bound by theory, it is believed that such an NMN sequestering agent would function to sequester NMN via a mechanism or route entirely independent of Nmnat and/or Nampt activity. One such example of a mechanism or route may include an agent which binds to another protein or chemical. A further example of a mechanism or route may include a metabolic reaction wherein an agent converts NMN into another molecule.

The term ‘neuroprotective’ as used herein refers to the ability to protect neurons or their axons or synapses in the central or peripheral nervous system from damage or death. Many different types of insult can lead to neuronal damage or death, for example: metabolic stress caused by hypoxia, hypoglycaemia, diabetes, loss of ionic homeostasis or other deleterious process, physical injury of neurons, exposure to toxic agents and numerous diseases affecting the nervous system including inherited disorders. It will be appreciated that this is only an illustrative list; many other examples will be found in the literature. The presence of an agent that is neuroprotective will enable a neuron to remain viable upon exposure to insults which may cause a loss of functional integrity in an unprotected neuron.

The term ‘medicament’ as used herein refers to a pharmaceutical formulation that is of use in treating, curing or improving a disease or in treating, ameliorating or alleviating the symptoms of a disease. A pharmaceutical formulation comprises a pharmacologically active ingredient in a form not harmful to the subject it is being administered to and additional constituents designed to stabilise the active ingredient and affect its absorption into the circulation or target tissue.

In one aspect of the invention, there is provided a pharmaceutical composition comprising a modulator as hereinbefore defined. In one embodiment, the pharmaceutical composition comprises a combination of an Nampt inhibitor and a Nmnat activator. Such an embodiment will provide the synergy of both inhibiting Nampt (i.e. reducing production of NMN) and activating Nmnat (i.e. increasing conversion of NMN to NAD).

The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19^{th} Edition, Gennaro, Ed., Mack Publishing Co., Easton, Pa., 1995.

Suitable pharmaceutical carriers include inert solid diluents or fillers, sterile aqueous solutions and various organic solvents. Examples of solid carriers are lactose, terra alba, sucrose, cyclodextrin, talc, gelatine, agar, pectin, acacia, magnesium stearate, stearic acid and lower alkyl ethers of cellulose. Examples of liquid carriers are syrup, peanut oil, olive oil, phospholipids, fatty acids, fatty acid amines, polyoxyethylene and water.

In one aspect of the invention, there is provided a method of treatment of a neurodegenerative disorder, such as a disorder involving axon degeneration comprising administering to a subject an NMN modulator.

In one aspect of the invention, there is provided a pharmaceutical composition comprising an NMN modulator for use in the treatment of a neurodegenerative disorder, such as a disorder involving axon degeneration.

Administration of NMN modulators according to the invention may be through various routes, for example oral, rectal, nasal, pulmonary, topical (including buccal and sublingual), transdermal, intraperitoneal, vaginal, parenteral (including subcutaneous, intramuscular, intradermal), intrathecal or intracerebroventricular. It will be appreciated that the preferred route will depend on the general condition and age of the subject to be treated, the nature of the condition to be treated and the active ingredient chosen.

Parenteral administration may be performed by subcutaneous, intramuscular, intraperitoneal or intravenous injection by means of a syringe, optionally a pen-like syringe. Alternatively, parenteral administration can be performed by means of an infusion pump. A further option is a formulation which may be a solution or suspension for the administration of the NMN modulator in the form of a nasal or pulmonal spray. As a still further option, the formulation containing the NMN modulator of the invention can also be adapted to transdermal administration, e.g. by needle-free injection or from a patch, optionally an iontophoretic patch, or transmucosal, e.g. buccal, administration.

NMN modulators of the current invention may be administered in several dosage forms, for example, as solutions, suspensions, emulsions, microemulsions, multiple emulsion, foams, salves, pastes, plasters, ointments, tablets, coated tablets, rinses, capsules, for example, hard gelatine capsules and soft gelatine capsules, suppositories, rectal capsules, drops, gels, sprays, powder, aerosols, inhalants, eye drops, ophthalmic ointments, ophthalmic rinses, vaginal pessaries, vaginal rings, vaginal ointments, injection solution, in situ transforming solutions, for example in situ gelling, in situ setting, in situ precipitating, in situ crystallization, infusion solution, and implants.

NMN modulators of the invention may further be compounded in, or attached to, for example through covalent, hydrophobic and electrostatic interactions, a drug carrier, drug delivery system and advanced drug delivery system in order to further enhance stability of the composition, increase bioavailability, increase solubility, decrease adverse effects, achieve chronotherapy well known to those skilled in the art, and increase patient compliance or any combination thereof.

Examples of carriers, drug delivery systems and advanced drug delivery systems include, but are not limited to, polymers, for example cellulose and derivatives, polysaccharides, for example dextran and derivatives, starch and derivatives, poly(vinyl alcohol), acrylate and methacrylate polymers, polylactic and polyglycolic acid and block co-polymers thereof, polyethylene glycols, carrier proteins, for example albumin, gels, for example, thermogelling systems, for example block co-polymeric systems well known to those skilled in the art, micelles, liposomes, microspheres, nanoparticulates, liquid crystals and dispersions thereof, L2 phase and dispersions thereof, well known to those skilled in the art of phase behaviour in lipid-water systems, polymeric micelles, multiple emulsions, self-emulsifying, self-microemulsifying, cyclodextrins and derivatives thereof, and dendrimers.

NMN modulators of the current invention may be useful in the composition of solids, semi-solids, powder and solutions for pulmonary administration, using, for example a metered dose inhaler, dry powder inhaler and a nebulizer, all being devices well known to those skilled in the art.

NMN modulators of the current invention may be useful in the composition of controlled, sustained, protracting, retarded, and slow release drug delivery systems. More specifically, but not limited to, modulators are useful in the composition of parenteral controlled release and sustained release systems (both systems leading to a many-fold reduction in number of administrations), well known to those skilled in the art. Even more preferably, are controlled release and sustained release systems administered subcutaneously. Without limiting the scope of the invention, examples of useful controlled release system and compositions are hydrogels, oleaginous gels, liquid crystals, polymeric micelles, microspheres and nanoparticles.

Methods to produce controlled release systems useful for compositions of the current invention include, but are not limited to, crystallization, condensation, co-crystallization, precipitation, co-precipitation, emulsification, dispersion, high pressure homogenisation, en-capsulation, spray drying, microencapsulating, coacervation, phase separation, solvent evaporation to produce microspheres, extrusion and supercritical fluid processes. General reference is made to Handbook of Pharmaceutical Controlled Release (Wise, D. L., ed. Marcel Dekker, New York, 2000) and Drug and the Pharmaceutical Sciences vol. 99: Protein Composition and Delivery (MacNally, E. J., ed. Marcel Dekker, New York, 2000).

NMN modulators are predicted to be of utility in the treatment of neurodegenerative disorders involving axon degeneration, such as Wallerian degeneration. Examples of disorders where such degeneration may be of importance include Alexander's disease, Alper's disease, Alzheimer's disease, Amyotrophic lateral sclerosis, Ataxia telangiectasia, Batten disease, Canavan disease, Cerebral palsy, Cockayne syndrome, Corticobasal degeneration, Creutzfeldt-Jakob disease, Diabetic neuropathy, Frontotemporal lobar degeneration, Glaucoma, Guillain-Barré syndrome, Hereditary spastic paraplegia, Huntington's disease, HIV associated dementia, Kennedy's disease, Krabbe's disease, Lewy body dementia, Motor neuron disease, Multiple System Atrophy, Multiple sclerosis, Narcolepsy, Neuroborreliosis, Niemann Pick disease, Parkinson's disease, Pelizaeus-Merzbacher Disease, Peripheral neuropathy, Pick's disease, Primary lateral sclerosis, Prion diseases, Progressive Supranuclear Palsy, Refsum's disease, Sandhoff's disease, Schilder's disease, Spinocerebellar ataxia, Spinal cord injury, Spinal muscular atrophy, Steele-Richardson-Olszewski disease, Stroke and other ischaemic disorders, Tabes dorsalis or Traumatic brain injury. This list is for illustrative purposes only and is not limiting or exhaustive. In one embodiment, the neurodegenerative disorder is selected from one or more of Alzheimer's disease, multiple sclerosis, Parkinson's disease, diabetic neuropathy, or an ophthalmic disorder such as glaucoma.

In one embodiment the modulator is intended for use as a neuroprotective medicament in the treatment of a neurodegenerative disorder resulting from neuronal injury.

In a further embodiment the modulator is intended for use as a neuroprotective medicament in the treatment of a neurodegenerative disorder involving axon degeneration (i.e. Wallerian degeneration) resulting from neuronal injury.

The term ‘injury’ as used herein refers to damage inflicted on the neuron, whether in the cell body or in axonal or dendritic processes. This can be a physical injury in the conventional sense i.e. traumatic injury to the brain, spinal cord or peripheral nerves caused by an external force applied to a subject. Other damaging external factors are for example environmental toxins such as mercury and other heavy metals, arsenic, pesticides and solvents. Alternatively, injury can result from an insult to the neuron originating from within the subject, for example: reduced oxygen and energy supply as in ischemic stroke and diabetic neuropathy, autoimmune attack as in multiple sclerosis or oxidative stress and free-radical generation as is believed to be important in amyotrophic lateral sclerosis. Injury is also used here to refer to any defect in the mechanism of axonal transport.

In another embodiment, the modulator is intended for use as a neuroprotective medicament wherein the neurodegenerative disorder is caused by a neuronal injury resulting from a disease.

In one embodiment, the neuronal injury results from trauma.

In one embodiment, the disorder is a neuronal injury induced by a chemotherapeutic agent. Certain drugs used in cancer chemotherapy such as Taxol, Velcade and vincristine, cause peripheral neuropathy which limits the maximum doses at which they can be used. Recent studies suggest that neurons suffering from Taxol or vincristine toxicity undergo Wallerian-like changes in their morphology and in the underlying molecular events. Inhibiting Wallerian degeneration could be particularly effective in this condition as neurons are only temporarily exposed to the neurotoxic agent. Simultaneous administration of Taxol or vincristine with an agent inhibiting Wallerian degeneration could therefore allow the drug to be used at substantially higher doses than is currently possible, in addition to drugs that inhibit Nampt further combating the cancer. Data is shown herein (Example 4) which demonstrates a neuroprotective effect from the Nampt inhibitor FK866 upon neurite degeneration caused by vincristine.

In another aspect of the invention there is provided the use of NMN, or a derivative, fragment or metabolite thereof, as a biomarker for axon degeneration, in particular Wallerian-like degeneration. Data is presented herein which shows that increased levels of NMN are likely to provide a diagnostically useful marker for the presence of axon degeneration, in particular Wallerian-like degeneration. The term ‘biomarker’ as used herein refers to a distinctive biological or biologically-derived indicator of a process, event or condition.

Biomarkers can be used in methods of diagnosis, e.g. clinical screening and prognosis assessment, in monitoring the results of therapy and in identifying patients most likely to respond to a particular therapeutic treatment as well as in drug screening and development. They can also be used in basic and medical research. Biomarkers and uses thereof are valuable for the identification of new drug treatments and for the discovery of new targets for drug treatment. In the present context, a biomarker can be replaced by a molecule, or measurable fragments of a molecule found upstream or downstream of the biomarker in a biological pathway.

In a further aspect of the invention there is provided a method for demonstrating axon degeneration, in particular Wallerian-like degeneration, comprising detecting and/or quantifying in a sample from a test subject, a biomarker as hereinbefore defined.

The term “detecting” as used herein refers to confirming the presence of the biomarker present in the sample. Quantifying the amount of the biomarker present in a sample may include determining the concentration of the biomarker present in the sample. Detecting and/or quantifying may be performed directly on the sample, or indirectly on an extract therefrom, or on a dilution thereof.

Detecting and/or quantifying can be performed by any method suitable to identify the presence and/or amount of a specific protein in a biological sample from a patient or a purification or extract of a biological sample or a dilution thereof. In methods of the invention, quantifying may be performed by measuring the concentration of the biomarker in the sample or samples.

Biological samples that may be tested in a method of the invention include tissue homogenates, tissue sections and biopsy specimens from a live subject, or taken post-mortem. The samples can be prepared, diluted or concentrated where appropriate, and stored in the usual manner. Biological samples can also include cerebrospinal fluid (CSF), whole blood, blood serum, plasma, urine, saliva, or other bodily fluid.

In one embodiment, detecting and/or quantifying is performed by one or more methods selected from SELDI (-TOF), MALDI (-TOF), a 1-D gel-based analysis, a 2-D gel-based analysis, Mass spec (MS), reverse phase (RP) LC, size permeation (gel filtration), ion exchange, affinity, HPLC, UPLC or other LC or LC-MS-based technique. Appropriate LC MS techniques include ICAT® (Applied Biosystems, CA, USA), or iTRAQ® (Applied Biosystems, CA, USA). Also enzymatic conversion of NMN into a molecule detectable by spectrophotometry or other methods.

Liquid chromatography (e.g. high pressure liquid chromatography (HPLC) or low pressure liquid chromatography (LPLC)), thin-layer chromatography, NMR (nuclear magnetic resonance) spectroscopy could also be used.

The biomarker may be directly detected, e.g. by SELDI or MALDI-TOF. Alternatively, the biomarker may be detected directly or indirectly via interaction with a ligand or ligands such as an antibody or a biomarker-binding fragment thereof, or other peptide, or ligand, e.g. aptamer, or oligonucleotide, capable of specifically binding the biomarker. The ligand may possess a detectable label, such as a luminescent, fluorescent or radioactive label, and/or an affinity tag.

In one embodiment, detecting and/or quantifying is performed using a biosensor, microanalytical, microengineered, microseparation or immunochromatography system.

The term ‘biosensor’ as used herein refers to something capable of detecting the presence of a biomarker. Using predictive biomarkers, appropriate diagnostic tools such as biosensors can be developed. The biosensor may incorporate an immunological method for detection of the biomarker(s), electrical, thermal, magnetic, optical (e.g. hologram) or acoustic technologies. Using such biosensors, it is possible to detect the target biomarker(s) at the anticipated concentrations found in biological samples.

In one embodiment, detecting and/or quantifying is performed by an immunological method. This may rely on an antibody, or a fragment thereof capable of specific binding to the biomarker. Suitable immunological methods include sandwich immunoassays, such as sandwich ELISA, in which the detection of the biomarker is performed using two antibodies which recognize different epitopes on a biomarker; radioimmunoassays (RIA), direct, indirect or competitive enzyme linked immunosorbent assays (ELISA), enzyme immunoassays (EIA), Fluorescence immunoassays (FIA), western blotting, immunoprecipitation, immunohistochemistry and any particle-based immunoassay (e.g. using gold, silver, or latex particles, magnetic particles, or Q-dots). Immunological methods may be performed, for example, in microtitre plate or strip format.

In one embodiment, detecting and/or quantifying is performed by an immunohistochemical method.

Immunological methods in accordance with the invention may be based, for example, on any of the following methods.

Immunoprecipitation is the simplest immunoassay method; this measures the quantity of precipitate, which forms after the reagent antibody has incubated with the sample and reacted with the target antigen present therein to form an insoluble aggregate. Immunoprecipitation reactions may be qualitative or quantitative.

In particle immunoassays, several antibodies are linked to the particle, and the particle is able to bind many antigen molecules simultaneously. This greatly accelerates the speed of the visible reaction. This allows rapid and sensitive detection of the biomarker.

In immunonephelometry, the interaction of an antibody and target antigen on the biomarker results in the formation of immune complexes that are too small to precipitate. However, these complexes will scatter incident light and this can be measured using a nephelometer. The antigen, i.e. biomarker, concentration can be determined within minutes of the reaction.

Radioimmunoassay (RIA) methods employ radioactive isotopes such as I¹²⁵ to label either the antigen or antibody. The isotope used emits gamma rays, which are usually measured following removal of unbound (free) radiolabel. The major advantages of RIA, compared with other immunoassays, are higher sensitivity, easy signal detection, and well-established, rapid assays. The major disadvantages are the health and safety risks posed by the use of radiation and the time and expense associated with maintaining a licensed radiation safety and disposal program. For this reason, RIA has been largely replaced in routine clinical laboratory practice by enzyme immunoassays.

Enzyme (EIA) immunoassays were developed as an alternative to radioimmunoassays (RIA). These methods use an enzyme to label either the antibody or target antigen. The sensitivity of EIA approaches that for RIA, without the danger posed by radioactive isotopes. One of the most widely used EIA methods for detection is the enzyme-linked immunosorbent assay (ELISA).

ELISA methods may use two antibodies one of which is specific for the target antigen and the other of which is coupled to an enzyme, addition of the substrate for the enzyme results in production of a chemiluminescent or fluorescent signal.

Fluorescent immunoassay (FIA) refers to immunoassays which utilize a fluorescent label or an enzyme label which acts on the substrate to form a fluorescent product. Fluorescent measurements are inherently more sensitive than colorimetric (spectrophotometric) measurements. Therefore, FIA methods have greater analytical sensitivity than EIA methods, which employ absorbance (optical density) measurement.

Chemiluminescent immunoassays utilize a chemiluminescent label, which produces light when excited by chemical energy; the emissions are measured using a light detector.

Immunological methods according to the invention can thus be performed using well-known methods. Any direct (e.g., using a sensor chip) or indirect procedure may be used in the detection of biomarkers of the invention.

The Biotin-Avidin or Biotin-Streptavidin systems are generic labelling systems that can be adapted for use in immunological methods of the invention. One binding partner (hapten, antigen, ligand, aptamer, antibody, enzyme etc) is labelled with biotin and the other partner (surface, e.g. well, bead, sensor etc) is labelled with avidin or streptavidin. This is conventional technology for immunoassays, gene probe assays and (bio)sensors, but is an indirect immobilisation route rather than a direct one. For example a biotinylated ligand (e.g. antibody or aptamer) specific for a biomarker of the invention may be immobilised on an avidin or streptavidin surface, the immobilised ligand may then be exposed to a sample containing or suspected of containing the biomarker in order to detect and/or quantify a biomarker of the invention. Detection and/or quantification of the immobilised antigen may then be performed by an immunological method as described herein.

In a further aspect of the invention there is provided a diagnostic kit for detecting axon degeneration, in particular Wallerian-like degeneration, comprising a biosensor configured to detect and/or quantify the biomarker as hereinbefore defined and instructions to use said kit in accordance with the methods as hereinbefore defined.

In one embodiment, the biosensor is an antibody. The term “antibody” as used herein includes, but is not limited to: polyclonal, monoclonal, bispecific, humanised or chimeric antibodies, single chain antibodies, Fab fragments and F(ab′)₂ fragments, fragments produced by a Fab expression library, anti-idiotypic (anti-Id) antibodies and epitope-binding fragments of any of the above. The term “antibody” as used herein also refers to immunoglobulin molecules and immunologically-active portions of immunoglobulin molecules, i.e., molecules that contain an antigen binding site that specifically binds an antigen. The immunoglobulin molecules of the invention can be of any class (e. g., IgG, IgE, IgM, IgD and IgA) or subclass of immunoglobulin molecule.

In another aspect of the invention there is provided a method of screening for an NMN modulator comprising the steps of:
a) blocking the synthesis of NMN, or a readily detectable version of NMN, in a biological sample;
b) incubating said sample with a test molecule; and
c) measuring the NMN-associated signal over time

such that a difference from the control decay curve is indicative of an NMN modulator.

It will be appreciated that a readily detectable version of NMN comprises a modification making it suitable for rapid quantification in a high-throughput system, for example by tagging the protein with a reporter such as a fluorescent protein. The synthesis of NMN can be blocked for example by using an inducible expression system, knocking down expression or adding a general protein synthesis inhibitor.

High-throughput screening technologies based on the biomarker, uses and methods of the invention, e.g. configured in an array format, are suitable to monitor biomarker signatures for the identification of potentially useful therapeutic compounds, e.g. ligands such as natural compounds, synthetic chemical compounds (e.g. from combinatorial libraries), peptides, monoclonal or polyclonal antibodies or fragments thereof, which may be capable of binding the biomarker.

Methods of the invention can be performed in array format, e.g. on a chip, or as a multiwell array. Methods can be adapted into platforms for single tests, or multiple identical or multiple non-identical tests, and can be performed in high throughput format. Methods of the invention may comprise performing one or more additional, different tests to confirm or exclude diagnosis, and/or to further characterise a condition.

In another aspect of the invention, there is provided an NMN modulator identified by a screening method as hereinbefore defined.

The invention will now be described, by way of example only, with reference to the accompanying examples:

### EXAMPLES

Materials and Methods

SCG Explant Cultures, Cut Injuries and FK866 Treatment

Superior cervical ganglia (SCG) were dissected from 0-2 days old mouse pups. Explants were placed into L15 (Leibovitz) medium (Invitrogen), cleaned from other tissues, cut in half and six half explants were placed in the centre of 3.5 cm tissue culture dishes pre-coated with poly-L-lysine (20 μg/ml for 2 h; Sigma) and laminin (20 μg/ml for 2 h; Sigma). Explants were cultured in DMEM containing 4500 mg/L glucose and 110 mg/L sodium pyruvate (Sigma), 2 mM glutamine, 1% penicillin/streptomycin, 1/50 B27 serum supplement and 100 ng/ml 7S NGF (all from Invitrogen) and 4 μM aphidicolin (Sigma) to block proliferation of non-neuronal cells. Neurites were allowed to extend for 7 days in all cultures before any treatment. After this time, neurites were separated from their cell bodies using a scalpel. FK866 (100 nM final concentration) was added to the growth medium one day before cut injuries, at the same time (time 0) or at 1 hour intervals up to 6 hours after the cut. In some experiments, NMN, NAD, Nicotinic acid adenine dinucleotide (NaAD) or Nicotinic acid (NA) were added to the culture medium at t=0 together with FK866. In another series of experiments, FK866 was added at t=0 and NMN or NAD were added to FK866 treated cultures several hours after cut injuries. Bright-field images were captured on an Olympus IX81 inverted microscope using a Soft Imaging Systems F-View camera linked to a PC running the Analysis software. Images of the same field of neurites were captured just after or at regular intervals up to 48-72 hours after cut injuries.

Results

### Example 1

### Effect of Nampt Inhibitor FK866 upon NAD Levels

This experiment analysed the effect of the Nampt inhibitor FK866 upon intracellular NAD(P)⁺ levels. In this experiment, FK866 (1-100 nM final concentration) was applied and the cultures were kept 8-72 hours in the presence of the drug. At this time, the explants were collected in 100 μl H₂O for NAD(P)⁺ determination as described in Billington et al. (2008) J Biol Chem 283(10), 6367-6374.

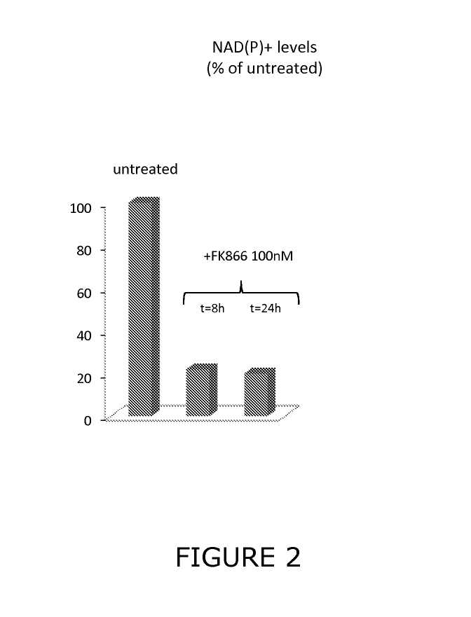

The results of this analysis can be seen in FIG. 2. It is known that FK866 is a potent inhibitor of Nampt which would therefore result in a reduction in the intracellular levels of the product NMN⁺. The results of this study clearly demonstrate that Nampt inhibition also results in depletion of NAD⁺ further along the NAD⁺ salvage pathway (as shown in FIG. 1). Therefore, the reduced intracellular levels of NMN⁺ result in a reduction in the turnover of NMN⁺ to NAD⁺ by isoforms of Nmnat. FIG. 2 illustrates NAD(P)⁺ levels (expressed as percent of untreated SCG cultures) in SCG cultures untreated or treated with 100 nM FK866 for 8 h or for 24 h.

### Example 2

### Effect of FK866 upon Cut Neurites

This experiment analysed the effect of the Nampt inhibitor FK866 upon cut neurites using the methodology described above. The results are shown in FIG. 3 which demonstrates that FK866 consistently mimics the Wld^{S} phenotype, preserving injured neurites in primary culture even if added shortly after cutting. In this experiment, SCG neurons were cultured for 7 days as described in the methodology, then the neurites were separated from the cell bodies by a scalpel, and the distal part of the neurites with respect to the cut was imaged immediately after the cut or 24 hours after the cut. In some SCG cultures, FK866 (100 nM final concentration) was added the day prior to the cut (right panels). The effect was observed to be shorter than for the Wld^{S} phenotype, possibly because NAD⁺ depletion has other negative effects, however, neurite survival was estimated to be increased by four fold in the presence of the Nampt inhibitor FK866.

### Example 3

### Effect of FK866 and NMN⁺ upon Cut Neurites

This experiment was performed in an analogous manner to that described in Example 2, with the exception that NMN⁺ was also added to the cut neurites in combination with the Nampt inhibitor FK866. The results of this study are shown in FIG. 4 where it can be seen that adding NMN⁺ to bypass Nampt, consistently reverts the protective effect of FK866 to delay Wallerian degeneration.

The results of these studies provide a strong link between the levels of NMN⁺ and Wallerian degeneration. For example, inhibition of Nampt with FK866 (known to reduce NMN⁺ levels) provided a neuroprotective effect upon cut neurites (FIG. 3) and addition of NMN⁺ to the Nampt inhibitor (i.e. increasing NMN⁺ levels) reverted the neuroprotective effect.

### Example 4

### Effect of FK866 and NMN⁺ upon Vincristine-Treated Neurites

This experiment was performed in an analogous manner to that described in Examples 2 and 3, however the neurites, instead of being separated by the cell body with a cut, were treated with the neurotoxic chemotherapy drug vincristine. In this experiment, SCG neurons were cultured for 7 days as described in the methodology, and then treated with 0.02 μM vincristine alone, with 0.02 μM vincristine plus 100 nM FK866 or with 0.02 μM vincristine plus 100 nM FK866 and 1 mM NMN. The distal part of the neurites was imaged immediately and at 24, 48 and 72 hours after adding the drugs.

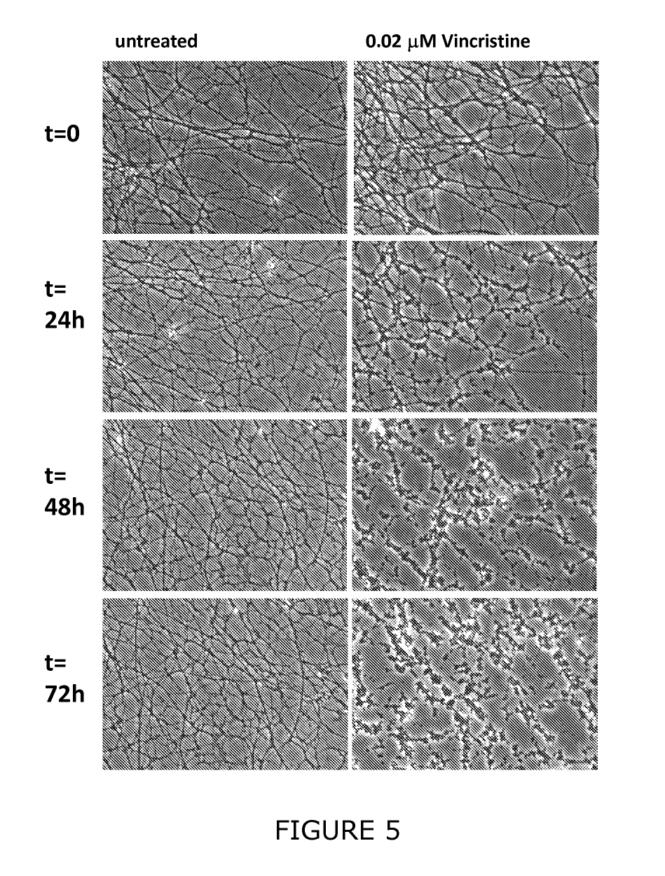

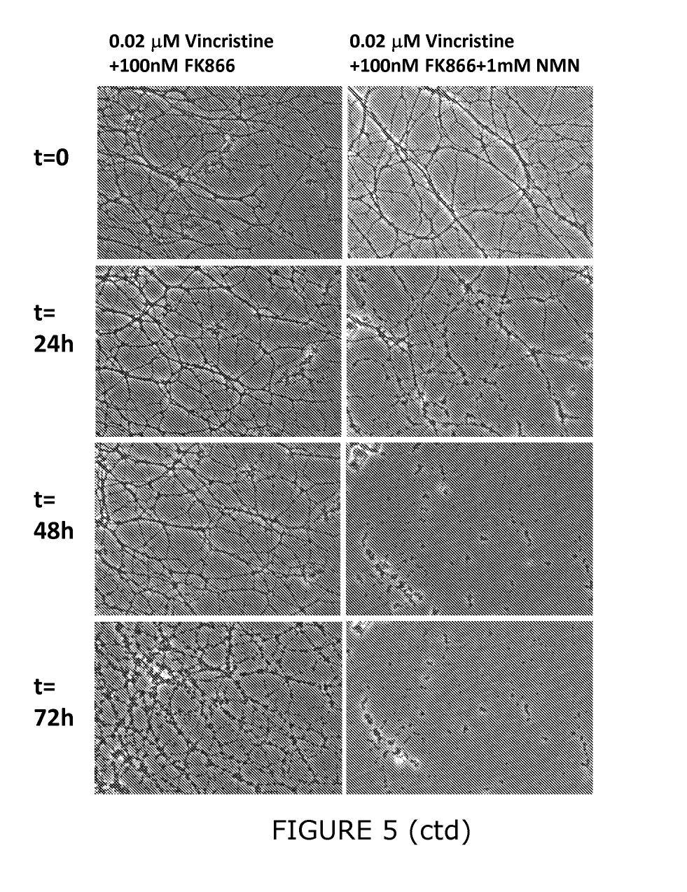

Vincristine causes a progressive distal-to-proximal degeneration of neurites. FIG. 5 shows the protection conferred by 100 nM FK866 on this toxic effect. The protective effect is reverted when 1 mM NMN is also added together with FK866.

1. Use of a nicotinamide mononucleotide (NMN) modulator as a neuroprotective medicament in the treatment of a neurodegenerative disorder.

2. Use as defined in claim 1, wherein the NMN modulator decreases NMN levels.

3. Use as defined in claim 1 or claim 2, wherein the NMN modulator is a Nampt inhibitor.

4. Use as defined in claim 3, wherein the Nampt inhibitor comprises N-[4-(1-benzoyl-4-piperidinyl)butyl]-3-(3-pyridinyl)-2E-propenamide (FK866) or N-(6-chlorophenoxyhexyl)-N′-cyano-N″-4-pyridylguanidine (CHS828).

5. Use as defined in claim 4, wherein the Nampt inhibitor comprises N-[4-(1-benzoyl-4-piperidinyl)butyl]-3-(3-pyridinyl)-2E-propenamide (FK866).

6. Use as defined in claim 1 or claim 2, wherein the NMN modulator is an Nmnat activator, such as an Nmnat2 activator.

7. Use as defined in claim 1 or claim 2, wherein the NMN modulator is an NMN sequestering agent.

8. Use as defined in any preceding claims, wherein the neurodegenerative disorder involves axon degeneration.

9. Use as defined in any of claims 1 to 7, wherein the neurodegenerative disorder involves Wallerian degeneration.

10. Use as defined in claim 9, wherein the Wallerian degeneration results from neuronal injury.

11. Use as defined in claim 10, wherein the neuronal injury results from disease, trauma or a chemotherapeutic agent.

12. Use as defined in any preceding claims wherein the neurodegenerative disorder is one or more of Alzheimer's disease, multiple sclerosis, Parkinson's disease, diabetic neuropathy, or an ophthalmic disorder such as glaucoma.

13. A pharmaceutical composition comprising a modulator as defined in any of claims 1 to 7.

14. A pharmaceutical composition as defined in claim 13, which comprises a combination of an Nampt inhibitor and an Nmnat activator.

15. Use of NMN, or a derivative, fragment or metabolite thereof, as a biomarker for axon degeneration, in particular Wallerian-like degeneration.

16. A method for demonstrating axon degeneration, in particular Wallerian-like degeneration, comprising, detecting and/or quantifying in a sample from a test subject, a biomarker as defined in claim 14 or claim 15.

17. A method as defined in claim 16 wherein detecting and/or quantifying is performed by one or more methods selected from SELDI (-TOF), MALDI (-TOF), a 1-D gel-based analysis, a 2-D gel-based analysis, Mass spec (MS), reverse phase (RP) LC, size permeation (gel filtration), ion exchange, affinity, HPLC, UPLC or other LC or LC-MS-based technique.

18. A method as defined in claim 17 wherein the detecting and/or quantifying is performed using a biosensor or a microanalytical, microengineered, microseparation or immunochromatography system.

19. A method as defined in claim 17 wherein detecting and/or quantifying using a biosensor is performed by an immunological method.

20. A diagnostic kit for detecting axon degeneration, in particular Wallerian-like degeneration, comprising a biosensor configured to detect and/or quantify the biomarker as defined in claim 14 or claim 15 and instructions to use said kit in accordance with the methods as defined in claims 16 to 19.

21. A method or diagnostic kit as defined in claim 19 or claim 20 wherein the biosensor is an antibody.

22. A method of screening for an NMN modulator comprising the steps of:
a) blocking the synthesis of NMN, or a readily detectable version of NMN, in a biological sample;
b) incubating said sample with a test molecule; and
c) measuring the NMN-associated signal over time,
such that a difference from the control decay curve is indicative of an NMN modulator.

23. An NMN modulator identified by a method as defined in claim 22.
